(19) United States
(12) Patent Application Publication
(10) Pub. No.: US 2013/0177906 A1
(43) Pub. Date: Jul. 11, 2013

(54) ENHANCED AMPLIFICATION OF TARGET NUCLEIC ACID

(71) Applicant: SAMSUNG TECHWIN CO., LTD., Changwon-city (KR)

(72) Inventor: Jun LI, Woodstock, MD (US)

(73) Assignee: SAMSUNG TECHWIN CO., LTD., Changwon-city (KR)

(21) Appl. No.: 13/721,937

(22) Filed: Dec. 20, 2012

Related U.S. Application Data
(60) Provisional application No. 61/583,860, filed on Jan. 6, 2012.

(51) Int. Cl. C12Q 1/68

(52) U.S. Cl. CPC C12Q 1/6876
USPC 435/6.11

(57) ABSTRACT

Described herein are a composition for an improved amplification of a target nucleic material, a kit containing the composition and a method for an improved amplification. The composition contains a primer sequence which has a ribonucleic acid segment which can be cleaved by a RNase activity. The method using such primer improves probe cleavage kinetics is to increase availability or duration of a single strand template for probe binding.

### FIELD

A combination of primer oligonucleotides for enhanced amplification or enrichment of a target nucleic acid in a sample, and a method of an enhanced amplification or enrichment of the target nucleic acid are described.

### BACKGROUND

Various processes for amplifying and detecting existing nucleic acid sequences in a sample are known. Amplification of a target nucleic acid in a sample may be used, for example, for diagnostic applications in particular, the target nucleic acid sequence may be only a small portion of the DNA or RNA in question.

Nucleic acid amplification can be accomplished by a variety of methods, including, but not being limited to, the polymerase chain reaction (PCR), nucleic acid sequence based amplification (NASBA), ligase chain reaction (LCR), isothermal amplification, and rolling circle amplification (RCA). The polymerase chain reaction (PCR) is the method most commonly used to amplify specific target DNA sequences.

The procedure of PCR is described in detail in U.S. Pat. Nos. 4,683,202, 4,683,195, 4,800,159, and 4,965,188, the contents of which are hereby incorporated herein in their entirety. Generally, the PCR process consists of introducing a molar excess of two or more extendable oligonucleotide primers to a reaction mixture comprising the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers

The ability to measure the kinetics of a PCR reaction by real-time detection has enabled accurate and precise determination of nucleic acid copy number with high sensitivity. This has become possible by detecting and measurement of PCR product during the amplification process by fluorescent dual-labeled hybridization probe technologies, such as the 5′ fluorogenic nuclease assay (“Taq-Man”) or endonuclease assay (“CataCleave”), discussed below.

In brief, the so-called CataCleave probe has a hybrid structure. A typical Catacleave probe has a DNA-RNA-DNA chimeric or hybrid structure, wherein both of 5′-end and 3′-end are labeled with a detectable marker. Upon binding to a target template, a ribnonuclease, for example RNase H enzyme, can recognize the hybrid structure and cleaves the RNA portion of the hybrid probe. Due to a dramatic Tm decrease upon cleavage, the fragmented probe dissociates from the template-probe complex and emits fluorescence.

Although the cleavage kinetics is significantly high, in reality it may be limited by several factors, primarily by template concentration and availability of a probe binding site. For example, during a Taq polymerase extension, the probe binding site of the template or the target sequence be sealed, leaving no space for probe binding. Therefore, for example, once a CataCleave probe is introduced to a real-time PCR reaction, the probe cleavage kinetics is controlled by PCR kinetics. That is, one cycle of PCR provides a narrow window for a probe to anneal to a single strand template or target sequence. This restriction applies to such a scenario that the probe binding site locates between a forward and a reverse primer.

### SUMMARY OF INVENTION

According an embodiment, a composition containing a first primer substantially complementary to a first part of a target sequence, a second primer substantially complementary to a second part of a target sequence, and a third primer which has the sequence of either the first primer or the second primer, except at least one internal region of 1-20 nucleotides of the third primer are corresponding ribonucleotides. Therefore, the third primer has a chimeric or hybrid structure of 5′-{DNA₁-RNA}n-DNA₂-3′, wherein n is an integer of 1-5, and may be comprised of 5-50 nucleotides. The probe, sharing the identical sequence with DNA₁, may be labeled with a detectable marker. The chimeric probe binding site is not flanked by the forward and the reverse primers.

In an embodiment, the composition further contains a first probe which has the sequence of the 5′-end segment of 5-50 nucleotides (or DNA₁) of one of the first primer or the second primer, except at least one internal region of 1-30 nucleotides are corresponding ribonucleotides. In an embodiment, the primers are of 15-100 nucleotides and the probe is of 5-50 nucleotides.

According to another embodiment, a method for amplifying a target sequence in a sample, including the steps of: (a) providing a sample to be tested for the presence of a target sequence; providing a pair of a first and a second amplification primers that are can anneal to the target sequence; (b) providing a probe comprising a detectable label and DNA and RNA nucleic acid sequences that are substantially complimentary to the target sequence; and (c) amplifying a PCR fragment with the first and second and/or third primers in the presence of an amplifying polymerase activity, amplification buffer; an RNase H activity and the probe and under conditions where the RNA sequences within the probe can form a RNA:DNA heteroduplex with the complimentary DNA sequences in the PCR fragment of the target sequence.

According to another embodiment, a method for detecting a target sequence in a sample, including the steps of: providing a sample to be tested for the presence of a target sequence; providing a pair of a first and a second and a third amplification primers that are can anneal to the target sequence; providing a probe comprising a detectable label and DNA and RNA nucleic acid sequences that are substantially complimentary to the target sequence; amplifying a PCR fragment with the first and second and/or third amplification primers in the presence of an amplifying polymerase activity, amplification buffer; an RNase H activity and the probe and under conditions where the RNA sequences within the probe can form a RNA:DNA heteroduplex with the complimentary DNA sequences in the PCR fragment of the target sequence; and detecting a real-time increase in the emission of a signal from the label on the probe, wherein the increase in signal indicates the presence of the target DNA in the sample.

The method may include, but is not limited to, regular PCR, real-time PCR, real-time reverse transcriptase PCR, or isothermal amplification,

In an embodiment, the real-time increase in the emission of the signal from the label on the probe results from the RNase H cleavage of the heteroduplex formed between the probe and one of the strands of the PCR fragment.

The DNA and RNA sequences of the probe are covalently linked.

The detectable label on the probe may be a fluorescent label, particularly a FRET pair.

The amplifying polymerase activity may be an activity of a thermostable DNA polymer. The RNase H activity may be the activity of a thermostable RNase H. The RNase H activity may be a hot start RNase H activity.

The sample may be a biological fluid including, but not limited to, blood, saliva, urine, etc. The sample may be a food sample or a food swipe.

The target sequence may be a genomic sequence of a pathogen, which may be bacteria, or virus, or a sequence of cancer, a polymorphism target, or genetic abnormality gene.

In an embodiment, there is provided a kit for the real-time detection of a target sequence in a sample, including: (a) a first primer substantially complementary to a first part of a target sequence, (b) a second primer substantially complementary to a second part of a target sequence, (c) a third primer which has the sequence of either the first primer or the second primer, except at least one internal region of 1-30 nucleotides of the third primer are corresponding ribonucleotides; (d) an amplifying polymerase activity, and (e) an RNase H activity. The third primer has a chimeric or hybrid structure of 5′-{DNA₁-RNA}n-DNA₂-3′, wherein n is an integer of 1-5, and may be comprised of 5-100 nucleotides. The kit may further have a fourth primer, which has the sequence of the other one of the first or the second primers, and optionally further have a second probe. The fourth primer and the second probe may have a chimeric structure of DNA-RNA-DNA.

In an embodiment, the Tm of the third primer may be in the range of 30-100° lower than that of the first primer or the second primer.

### BRIEF DESCRIPTION OF DRAWINGS

All publications and patent applications mentioned in this specification are herein incorporated by reference to the same extent as if each individual publication or patent application was specifically and individually indicated to be incorporated by reference.

FIG. 1 is an illustration explaining the mechanism of enhanced CataCleave reaction according to an embodiment.

### DETAILED DESCRIPTION

The practice of the embodiments described herein employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

As used herein, the term “nucleic acid” refers to an oligonucleotide or polynucleotide, wherein said oligonucleotide or polynucleotide may be modified or may comprise modified bases. Oligonucleotides are single-stranded polymers of nucleotides comprising from 2 to 60 nucleotides. Polynucleotides are polymers of nucleotides comprising two or more nucleotides. Polynucleotides may be either double-stranded DNAs, including annealed oligonucleotides wherein the second strand is an oligonucleotide with the reverse complement sequence of the first oligonucleotide, single-stranded nucleic acid polymers comprising deoxythymidine, single-stranded RNAs, double stranded RNAs or RNA/DNA heteroduplexes. Nucleic acids include, but are not limited to, genomic DNA, cDNA, hnRNA, snRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample. Nucleic acids may be composed of a single type of sugar moiety, e.g., as in the case of RNA and DNA, or mixtures of different sugar moieties, e.g., as in the case of RNA/DNA chimeras.

A “target DNA or “target RNA”” or “target nucleic acid,” or “target nucleic acid sequence” or “target sequence” refers to a nucleic acid that is targeted by DNA amplification. A target nucleic acid sequence serves as a template for amplification in a PCR reaction or reverse transcriptase-PCR reaction. Target nucleic acid sequences may include both naturally occurring and synthetic molecules. Exemplary target nucleic acid sequences include, but are not limited to, genomic DNA or genomic RNA.

As used herein, “label” or “detectable label” can refer to any chemical moiety attached to a nucleotide, nucleotide polymer, or nucleic acid binding factor, wherein the attachment may be covalent or non-covalent. Preferably, the label is detectable and renders said nucleotide or nucleotide polymer detectable to the practitioner of the invention. Detectable labels include luminescent molecules, chemiluminescent molecules, fluorochromes, fluorescent quenching agents, colored molecules, radioisotopes or scintillants. Detectable labels also include any useful linker molecule (such as biotin, avidin, streptavidin, HRP, protein A, protein G, antibodies or fragments thereof, Grb2, polyhistidine, Ni²⁺, FLAG tags, myc tags), heavy metals, enzymes (examples include alkaline phosphatase, peroxidase and luciferase), electron donors/acceptors, acridinium esters, dyes and calorimetric substrates. It is also envisioned that a change in mass may be considered a detectable label, as is the case of surface plasmon resonance detection. The skilled artisan would readily recognize useful detectable labels that are not mentioned above, which may be employed in the operation of the present invention

As used herein, the term “oligonucleotide,” “oligomner,” or “oligo” is used sometimes interchangeably with “primer.” The term “primer” refers to an oligonucleotide that acts as a point of initiation of DNA synthesis in a PCR reaction. A primer is usually about 15 to about 100 nucleotides in length and hybridizes to a target region complementary to the target sequence. In an embodiment, primers are of 20-30 nucleotides. In another embodiment, primers are composed of 20-26 nucleotides.

Oligonucleotides may be synthesized and prepared by any suitable methods (such as chemical synthesis), which are known in the art. Oligonucleotides may also be conveniently available through commercial sources.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

As used herein, the term “substantially complementary” refers to two nucleic acid strands that are sufficiently complimentary in sequence to anneal and form a stable duplex. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from one to many mismatches so long as the hybridization conditions are sufficient to allow, for example discrimination between a pairing sequence and a non-pairing sequence. Accordingly, “substantially complementary” sequences can refer to sequences with base-pair complementarity of 100, 95, 90, 80, 75, 70, 60, 50 percent or less, or any number in between, in a double-stranded region.

A person of skill in the art will know how to design PCR primers flanking a target sequence of interest. Synthesized primers are typically between 15 and 35 base pairs, 20 and 30 base pairs, or 20-26 base pairs in length with a melting temperature, T_{M} of around 55° C.

As used herein, the term “PCR fragment” or “amplicon” refers to a polynucleotide molecule (or collectively the plurality of molecules) produced following the amplification of a particular target nucleic acid. A PCR fragment is typically, but not exclusively, a DNA PCR fragment. A PCR fragment can be single-stranded or double-stranded, or in a mixture thereof in any concentration ratio. A PCR fragment can be 100-500 nucleotides or more in length.

An amplification “buffer” is a compound added to an amplification reaction which modifies the stability, activity, and/or longevity of one or more components of the amplification reaction by regulating the amplification reaction. The buffering agents of the invention are compatible with PCR amplification and RNase H cleavage activity. Examples of buffers include, but are not limited to, HEPES ((4-(2-hydroxyethyl)-1-piperazineethanesulfonic acid), MOPS (3-(N-morpholino)-propanesulfonic acid), and acetate or phosphate containing buffers and the like. In addition, PCR buffers may generally contain up to about 70 mM KCl and about 1.5 mM or higher MgCl₂, to about 50-200 μM each of dATP, dCTP, dGTP and dTTP. The buffers of the invention may contain additives to optimize efficient reverse transcriptase-PCR or PCR reactions.

An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. Additives may be optionally added to improve selectivity of primer annealing provided the additives do not interfere with the activity of RNase H.

As used herein, the term “thermostable,” as applied to an enzyme, refers to an enzyme that retains its biological activity at elevated temperatures (e.g., at 55° C. or higher), or retains its biological activity following repeated cycles of heating and cooling. Thermostable polynucleotide polymerases find particular use in PCR amplification reactions.

As used herein, a “thermostable polymerase” is an enzyme that is relatively stable to heat and eliminates the need to add enzyme prior to each PCR cycle. Non-limiting examples of thermostable polymerases may include polymerases isolated from the thermophilic bacteria Thermus aquaticus (Taq polymerase), Therms thermophilus (Tth polymerase), Thermococcus litoralis (Tli or VENT polymerase), Pyrococcus furiosus (Pfu or DEEPVENT polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME polymerase) Thermotoga neapolitana (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq polymerase. Many variants of Taq polymerase with enhanced properties are known and include AmpliTaq, AmpliTaq Stoffel fragment, SuperTaq, SuperTaq plus, LA Taq, LApro Taq, and EX Taq.

### Reverse Transcriptase—PCR Amplification of a RNA Target Nucleic Acid Sequence

One of the most widely used techniques to study gene expression exploits first-strand cDNA for mRNA sequence(s) as template for amplification by the PCR. This method, often referred to as reverse transcriptase-PCR, exploits the high sensitivity and specificity of the PCR process and is widely used for detection and quantification of RNA.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” reverse transcriptase PCR methods use a common buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV reverse transcriptase and Taq DNA Polymerase wherein the initial 65° C. RNA denaturation step was omitted.

The first step in real-time, reverse-transcription PCR is to generate the complementary DNA strand using one of the template specific DNA primers. In traditional PCR reactions this product is denatured, the second template specific primer binds to the cDNA, and is extended to form duplex DNA. This product is amplified in subsequent rounds of temperature cycling. To maintain the highest sensitivity it is important that the RNA not be degraded prior to synthesis of cDNA. The presence of RNase H in the reaction buffer will cause unwanted degradation of the RNA:DNA hybrid formed in the first step of the process because it can serve as a substrate for the enzyme. There are two major methods to combat this issue. One is to physically separate the RNaseH from the rest of the reverse-transcription reaction using a barrier such as wax that will melt during the initial high temperature DNA denaturation step. A second method is to modify the RNase H such that it is inactive at the reverse-transcription temperature, typically 45-55° C. Several methods are known in the art, including reaction of RNase H with an antibody, or reversible chemical modification. For example, a hot start RNase H activity as used herein can be an RNase H with a reversible chemical modification produced after reaction of the RNase H with cis-aconitic anhydride under alkaline conditions. When the modified enzyme is used in a reaction with a Tris based buffer and the temperature is raised to 95° C. the pH of the solution drops and RNase H activity is restored. This method allows for the inclusion of RNase H in the reaction mixture prior to the initiation of reverse transcription.

Additional examples of RNase H enzymes and hot start RNase H enzymes that can be employed in the invention are described in U.S. Patent Application Publication No. 2009/0325169 to Walder et al.

### Real-Time PCR of a Target Nucleic Acid Sequence Using a CataCleave Probe

Post-amplification amplicon detection is both laborious and time consuming. Real-time methods have been developed to monitor amplification during the PCR process. These methods typically employ fluorescently labeled probes that bind to the newly synthesized DNA or dyes whose fluorescence emission is increased when intercalated into double stranded DNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red. Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons, TaqMan probes (e.g., U.S. Pat. Nos. 5,210,015 and 5,487,972), and CataCleave probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded the distance between the donor and acceptor chromophores increases such that FRET is reversed and donor emission can be monitored using specialized instrumentation. TaqMan and CataCleave technologies differ from the molecular beacon in that the FRET probes employed are cleaved such that the donor and acceptor chromophores become sufficiently separated to reverse FRET. The entire contents of U.S. Pat. No. 5,763,181 is incorporated herein by reference.

TaqMan technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan probe binds to one strand of the amplicon at the same time that the primer binds. As the DNA polymerase extends the primer the polymerase will eventually encounter the bound TaqMan probe. At this time the exonuclease activity of the polymerase will sequentially degrade the TaqMan probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan works a specific amplicon can be detected only once for every cycle of PCR. Extension of the primer through the TaqMan target site generates a double stranded product that prevents further binding of TaqMan probes until the amplicon is denatured in the next PCR cycle.

U.S. Pat. No. 5,763,181, the content of which is incorporated herein by reference, describes another real-time detection method (referred to as “CataCleave”). CataCleave technology differs from TaqMan in that cleavage of the probe is accomplished by a second enzyme that does not have polymerase activity. The CataCleave probe has a sequence within the molecule which is a target of an endonuclease, such as, for example a restriction enzyme or RNase. In one example, the CataCleave probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes an RNase H enzyme that will specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan probe and donor emission can be monitored. Cleavage and dissociation regenerates a site for further CataCleave binding. In this way it is possible for a single amplicon to serve as a target or multiple rounds of probe cleavage until the primer is extended through the CataCleave probe binding site.

### Labeling of a Target-Specific CataCleave Probe

The term “probe” comprises a polynucleotide that comprises a specific portion designed to hybridize in a sequence-specific manner with a complementary region of a specific nucleic acid sequence, e.g., a target nucleic acid sequence. In one embodiment, the oligonucleotide probe is in the range of 12-30 nucleotides in length. In another embodiment, the oligonucleotide probe is in the range of 13-16 nucleotides in length.

The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices can be found in many of the references describing Taq-man assays or CataCleave, described in U.S. Pat. Nos. 5,763,181, 6,787,304, and 7,112,422, the contents of which contents are incorporated herein by reference in their entirety.

As used herein, a “label” or “detectable label” may refer to any label of a CataCleave probe comprising a fluorochrome compound that is attached to the probe by covalent or non-covalent means.

As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides light that is absorbed by a fluorescence acceptor. The term “fluorescent acceptor” or “fluorescence acceptor” refers to either a second fluorochrome or a quenching molecule that absorbs energy emitted from the fluorescence donor. The second fluorochrome absorbs the energy that is emitted from the fluorescence donor and emits light of longer wavelength than the light emitted by the fluorescence donor. The quenching molecule absorbs energy emitted by the fluorescence donor.

Any luminescent molecule, preferably a fluorochrome and/or fluorescent quencher may be used in the practice of this invention, including, for example, Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY FL, BODIPY 630/650, BODIPY 6501665, BODIPY TMR-X, BODIPY TR-X, Dialkylaminocoumarin, Cy5.5, Cy5, Cy3.5, Cy3, DTPA(Eu³⁺)-AMCA and TTHA(Eu³⁺)AMCA.

In one embodiment, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ position of the probe.

In one embodiment, reporter molecules are fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. For example, in a preferred embodiment of the invention, the quencher molecule is non-fluorescent. Generally whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange; N-(p-(2-benzoxazolyl)phenyl)maleimide; benzoxadiazoles, stilbenes, pyrenes, and the like.

In one embodiment, reporter and quencher molecules are selected from fluorescein and non-fluorescent quencher dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like.

Rhodamine and non-fluorescent quencher dyes are also conveniently attached to the 3′ end of an oligonucleotide at the beginning of solid phase synthesis, e.g., Woo et al., U.S. Pat. No. 5,231,191; and Hobbs, Jr., U.S. Pat. No. 4,997,928.

### Real-Time Detection of Target Nucleic Acid Sequences Using a CataCleave Probe

The labeled oligonucleotide probe may be used as a probe for the real-time detection of target nucleic acid sequence in a sample.

A CataCleave oligonucleotide probe is first synthesized with DNA and RNA sequences that are complimentary to sequences found within a PCR amplicon comprising a selected target sequence. In one embodiment, the probe is labeled with a FRET pair, for example, a fluorescein molecule at one end of the probe and a non-fluorescent quencher molecule at the other end. Hence, upon hybridization of the probe with the PCR amplicon, a RNA:DNA heteroduplex forms that can be cleaved by an RNase H activity.

RNase H hydrolyzes RNA in RNA-DNA hybrids. This enzyme was first identified in calf thymus but has subsequently been described in a variety of organisms. RNase H activity appears to be ubiquitous in eukaryotes and bacteria. Although RNase H's constitute a family of proteins of varying molecular weight and nucleolytic activity, substrate requirements appear to be similar for the various isotypes. For example, most RNase H's studied to date function as endonucleases and requiring divalent cations (e.g., Mg²⁺, Mn²⁺) to produce cleavage products with 5′ phosphate and 3′ hydroxyl termini.

RNase HI from E. coli is the best-characterized member of the RNase H family.

In addition to RNase HI, a second E. coli RNase H, RNase HII has been cloned and characterized (Itaya, M., Proc. Natl. Acad. Sci. USA, 1990, 87, 8587-8591). It is comprised of 213 amino acids while RNase HI is 155 amino acids long. E. coli RNase HIM displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase HI enzymes are reported to have molecular weights in the 68-90 kDa range, be activated by either Mn²⁺ or Mg²⁺ and be insensitive to sulfhydryl agents. In contrast, RNase H II enzymes have been reported to have molecular weights ranging from 31-45 kDa, to require Mg²⁺ to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108.).

An enzyme with RNase HII characteristics has been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

According to an embodiment, real-time nucleic acid amplification is performed on a target polynucleotide in the presence of a thermostable nucleic acid polymerase, an RNase H activity, a pair of PCR amplification primers capable of hybridizing to the target polynucleotide, a third primer which has a sequence identical to one of the PCR amplification primers except that at least one region of 2-5 nucleotides in the sequence are corresponding ribonucleotides, and the labeled CataCleave oligonucleotide probe. During the real-time PCR reaction, cleavage of the probe by RNase H leads to the separation of the fluorescent donor from the fluorescent quencher and results in the real-time increase in fluorescence of the probe corresponding to the real-time detection of target DNA sequences in the sample.

In certain embodiments, the real-time nucleic acid amplification permits the real-time detection of a single target DNA molecule in less than about 40 PCR amplification cycles as shown in FIG. 7.

### Kits

The disclosure herein also provides for a kit format which comprises a package unit having one or more reagents for the real-time detection of target nucleic acid sequences in a sample. The kit may also contain one or more of the following items: buffers, instructions, and positive or negative controls. Kits may include containers of reagents mixed together in suitable proportions for performing the methods described herein. Reagent containers preferably contain reagents in unit quantities that obviate measuring steps when performing the subject methods.

Kits may also contain reagents for real-time PCR including, but not limited to, a thermostable polymerase, thermostable RNase H, a pair of primers selected to amplify a target nucleic acid target sequence, a labeled CataCleave oligonucleotide probe that anneals to the real-time PCR product and allow for the detection of target nucleic acid sequences according to the methodology described herein, and a third primer which has a sequence identical to one of the PCR amplification primers except that at least one region of 2-5 nucleotides in the sequence are corresponding ribonucleotides. Kits may comprise reagents for the detection of two or more target nucleic acid sequences. In another embodiment, the kit reagents further comprised reagents for the extraction of genomic DNA or RNA from a biological sample. Kit reagents may also include reagents for reverse transcriptase-PCR analysis where applicable.

An amplification of a target sequence may be performed in a suitable reaction buffer containing, for example, thermostable RNase H, thermostable DNA polymerase, and Uracil-N-Glycosylase (UNG). During the reaction cycle the sample is first incubated at 37° C. so that the Uracil-N-Glycosylase can cleave any nucleic acid containing uracil that was carried over from previous assays. This material is a contaminant that will result in false positive detection of a target. UNG does not affect the nucleic acids recovered from the bacterial lysis as it does not contain uracil in place of thymidine. After incubation, the nucleic acids recovered from the lysis is denatured at high temperature and the UNG is inactivated. As the temperature is lowered, target gene-specific primers including a pair of primers and at least one chimeric primers which have a same sequence as the primers except at least one region of 2-5 nucleotides are corresponding ribonucleotides, and CataCleave probe hybridize to any target species-specific nucleic acid. After hybridization, the CataCleave probe can be cleaved by the action of RNase H, which cleaves the RNA portion of a RNA/DNA duplex. Once cleaved, the probe fragments dissociate from their target and diffuse into the reaction buffer. The CataCleave probe is labeled with a fluorescent donor and quencher so that in the intact state fluorescence from the donor is greatly reduced. Diffusion of the probe fragments increases the distance between the donor and quencher such that the donor fluorescence is no longer attenuated. The resulting increase in donor fluorescence emission can be detected in real-time using a suitable instrument, such as the Applied Biosystems 7500 Fast Real-Time PCR System or the Biorad CFX96 real-time PCR thermocycler. After the probe fragments dissociate from the target, another CataCleave probe can hybridize in the same location and the cleavage reaction is repeated. This cyclic process results in signal amplification as a single amplicon can serve as template for cleavage of multiple CataCleave probes. During this time the gene specific primers are also extended by the DNA polymerase in the presence of nucleoside triphosphates. Once the primers extend over the site for CataCleave probe binding no further cleavage can occur. After extension, the cycle of amplification and detection is completed and the number of amplicons has doubled. These newly synthesized amplicons then serve as template in the next amplification/detection cycle.

In an embodiment, in order to improve probe cleavage kinetics, a strategy of increasing availability (or duration) of a single strand template for probe binding is proposed. This can be realized by moving the probe binding site in between the primer pair to upstream of 5′ end of either (or both) of the primers. In addition, at least one of the primers is modified to have a structure of 5′-DNA₁-RNA-DNA₂-3′ structure, wherein DNA₁ is a DNA sequence or a DNA-RNA-DNA sequence. The primer(s) of modified structure to have a chimeric structure or a DNA₁-RNA-DNA₂ structure is referred to herein as “chimeric primer,” or “third primer.” The chimeric primer has a same sequence as one of the pair of primers, except at least one internal region of 1-30 nucleotides are corresponding ribonucletodies. The term “internal region” is used to indicate that the chimeric primer(s) has DNA sequences at both 5′- and 3′-ends.

Upon primer binding to the target, the 5′-DNA₁ portion will be removed after RNase H cleavage, leaving the remaining RNA-DNA₂ primer intact for polymerase recognition and extension and a single-stranded free site complementary to the DNA₁ region on the target template. This free single stranded site allows binding of a probe, for example a CataCleave probe that shares the exact same sequence as DNA₁. Furthermore, since the probe annealing region remains as a single stranded template throughout PCR cycles, this allows multiple unrestricted CataCleave reactions.

The composition and method according to embodiments can improve CataCleave reactions kinetics in real-time PCR. Given adequate template concentration, the CataCleave reaction shall then not be limited by PCR.

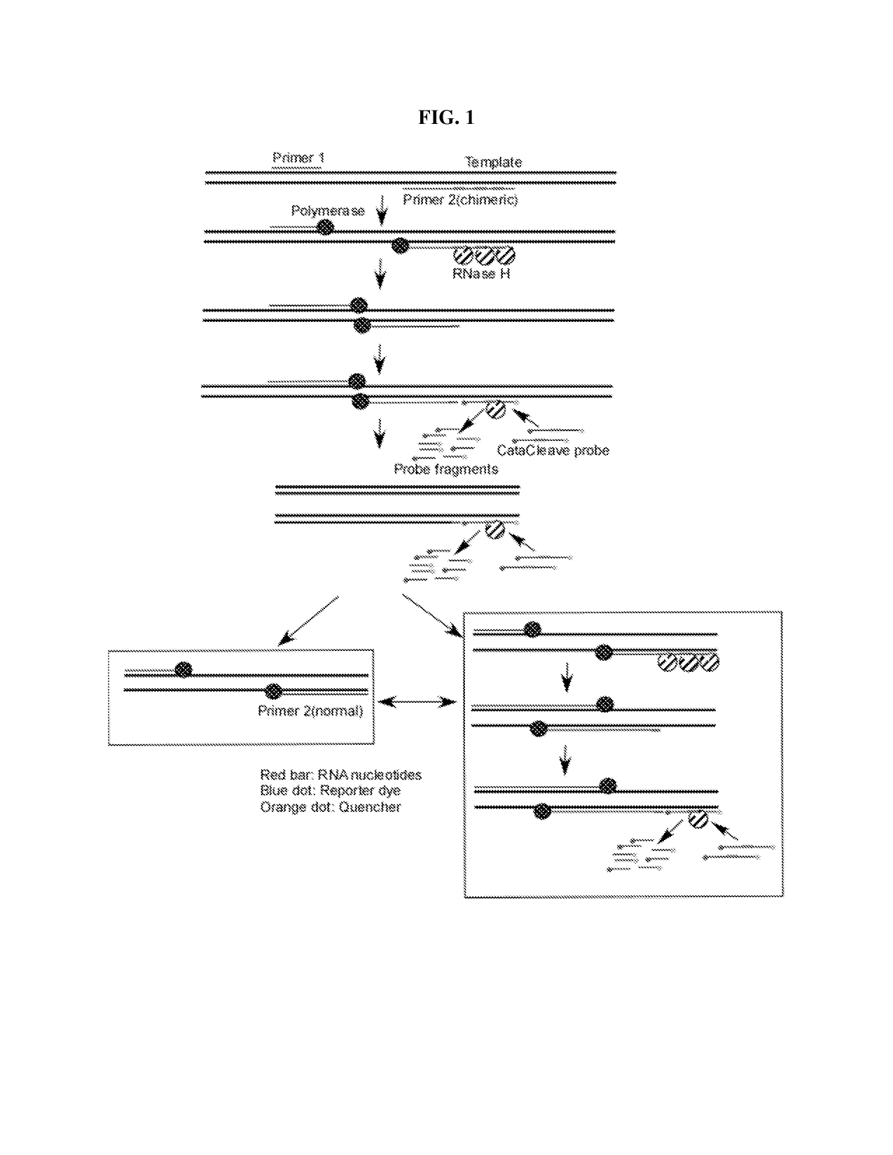

FIG. 1 illustrates a schematic view of the method according to an embodiment. In FIG. 1, the reaction contains at least three primers (i.e., first (or forward) primer, second (or reverse) primer, and a first chimeric primer) and one CataCleave probe. The primer set includes one forward primer, one reverse primer, and one chimeric primer that shares the exact sequence as forward or reverse primer. For example, Primer 1 as a normal sequence (e.g., forward primer), Primer 2 as a normal sequence (e.g., reverse primer; named Primer 2-Normal), and another Primer 2 as a chimeric sequence (e.g., chimeric reverse primer; named Primer 2-Chimeric). The function of Primer 2-Normal is solely to generate intact double strand DNA template; whereas Primer 2-Chimeric leads to a double strand DNA with 3′ overhang as a single strand template for CataCleave reactions.

During PCR, Primer 2-Normal and Primer 2-Chimeric compete for the annealing site. Since both shared the same sequence, each theoretically has a 50% chance. Annealing of Primer 2-Normal triggers regular polymerase binding and extension. The 5′ end of the newly-synthesized strand remains intact, as a template for the next round of PCR. However, if Primer 2-Chimeric binds to the annealing site, two reactions will be initiated simultaneously: (i) DNA polymerase binds to the structure and starts to extend the sequence at 3′ end, and (ii), due to the presence of DNA-RNA-DNA structure in Primer 2-Chimeric, RNase H recognize it and cleaves in the RNA region, generating two fragments, 5′-DNA₁ and 3′-DNA₂. Tm of each fragment must be carefully controlled so that, after cleavage, 3′-DNA₂ remains binding whereas 5′-DNA₁ detaches. 5′-DNA₁ region may be in a chimeric structure of DNA-RNA-DNA to ensure the whole piece dissociate from the template.

After removal of 5′-DNA₁, the 3′ end of the template is left as an over-hang. This provides a single strand template for a CataCleave probe. The CataCleave probe must share the same sequence as 5′-DNA₁ of Primer 2-Chimeric (or shorter, if necessary) and may contain modified nucleotides, such as LNA or PNA, to boost its Tm. Throughout each cycle of PCR, the 3′ overhang remains open and is not sealed by polymerase extension. Since modified probe has higher Tm than that of the fragment DNA₁, annealing of DNA₁ except for the probe back to the target sequence is therefore impossible. Or, DNA₁ is cleaved in multiple fragments by RNase HII and therefore each piece has very minimal or no chance to re-annealing due to their low Tm. These mechanisms shall dramatically increase availability of single-stranded template for CataCleave reactions, and much higher cleavage kinetics as well.

A single reaction may contain 3 or 4 primers, and 1 or 2 CataCleave probes. Among them, a pair of normal primer sequences, and 1 or 2 chimeric primers. For example, a reaction may contain the following oligonucleotides (RNA residues are underlined):

Primer 1-Normal:
ATTCTACGGCTACGTTAGTCGTCGTGCGGCGTG
Primer 1-Chimeric:
ATTCUACGGGTACGUUAGTCGTCGTGCGGCGTG
Probe 1:
ATTCTACGGGTACGT
Primer 2-Normal:
CGTGACTGGTAGAACGTAATCGGAACTGCGACGT
Primer 2-Chimeric:
CGTGACUGGTAGAACGTAATCGGAACTGCGACGT
Probe 2:
CGTGACUGGTAGAA

The “Normal” primers are used to generate full-length and intact amplicon. However, the “Chimeric” primers are used not only to initiate polymerase extension, but also provide their 5′ end as template for RNase H cleavage. The resulting overhangs are therefore open for multiple runs of probe binding and cleavage, free of PCR interference.

It is expected that the combination of primers and a probe accordance with an embodiment can have faster CataCleave kinetics since a single-stranded template is always open for probe binding and cleavage.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

1-19. (canceled)

20. A composition comprising
(a) a first primer substantially complementary to a first region of a target sequence;
(b) a second primer substantially complementary to a complementary sequence of a second region of the target sequence;
(c) a third primer of the sequence of one the first primer or the second primer, except at least one internal region of 1-30 nucleotides are corresponding ribonucleotides, wherein the third primer has a structure of 5′-{DNA₁-RNA}n-DNA₂-3′, wherein n is an integer of 1-5; and
(d) a first probe which has the sequence of the 5′-end region of one of the first primer or the second primer, except at least one internal region of 1-30 nucleotides are corresponding ribonucleotides, said first probe comprising a detectable label.

21. The composition according to claim 20, which further comprises a fourth primer which consists of the sequence of the other of the first primer or the second primer, except at least one internal region of 1-30 nucleotides are corresponding ribonucleotides, wherein the third primer has a structure of 5′-{DNA₁-RNA}n-DNA₂-3′, wherein n is an integer of 1-5.

22. The composition according to claim 20, which further comprises a second probe which has the sequence of the 5′-end region of the other of the first primer or the second primer, except at least one internal region of 1-30 nucleotides are corresponding ribonucleotides, said second probe comprising a detectable label.

23. The composition according to claim 20, wherein the DNA and RNA sequences of the probe are covalently linked.

24. The composition according to claim 20, wherein the detectable label on the probe may be a fluorescent label.

25. A method for amplifying a target sequence in a sample, including the steps of:
(a) providing a sample to be tested for the presence of a target sequence;
(b) providing a pair of a first and a second amplification primers that are can anneal to the target sequence and a third primer of the sequence of one the first primer or the second primer, except at least one internal region of 1-30 nucleotides are corresponding ribonucleotides, wherein the third primer has a structure of 5′-{DNA₁-RNA}n-DNA₂-3′, wherein n is an integer of 1-5;
(c) providing a first probe which has the sequence of the 5′-end region of 5-50 nucleotides of one of the first primer or the second primer, except at least one internal region of 1-30 nucleotides are corresponding ribonucleotides, said first probe comprising a detectable label; and
(d) amplifying a PCR fragment between the first and second amplification primers in the presence of an amplifying polymerase activity, amplification buffer; an RNase H activity and the probe and under conditions where the RNA sequences within the probe can form a RNA:DNA heteroduplex with the complimentary DNA sequences in the PCR fragment of the target sequence.

26. The method according to claim 25, in which the step (b) further comprises providing a fourth primer which consists of the sequence of the other of the first primer or the second primer, except at least one internal region of 1-30 nucleotides are corresponding ribonucleotides, wherein the third primer has a structure of 5′-DNA₁-RNA-DNA₂-3′, wherein DNA₁ is a DNA sequence or a DNA-RNA-DNA sequence.

27. The method according to claim 25, in which the step (c) further comprises providing a second probe which has the sequence of the 5′-end region of 5-50 nucleotides of the other of the first primer or the second primer, except at least one internal region of 1-30 nucleotides are corresponding ribonucleotides, said second probe comprising a detectable label.

28. The method according to claim 25, wherein the amplifying polymerase activity is an activity of a thermostable DNA polymer.

29. The method according to claim 25, wherein the RNase H activity is the activity of a thermostable RNase H.

30. A method for the real-time detection of a target sequence in a sample, comprising the steps of:
(a) providing a sample to be tested for the presence of a target sequence;
(b) providing a pair of a first and a second amplification primers that are can anneal to the target sequence and a third primer of the sequence of one the first primer or the second primer, except at least one internal region of 1-30 nucleotides are corresponding ribonucleotides, wherein the third primer has a structure of 5′-{DNA₁-RNA}n-DNA₂-3′, wherein n is an integer of 1-5;
(c) providing a first probe which has the sequence of the 5′-end region of 5-50 nucleotides of one of the first primer or the second primer, except at least one internal region of 1-30 nucleotides are corresponding ribonucleotides, said first probe comprising a detectable label;
(d) amplifying a PCR fragment between the first and second amplification primers in the presence of an amplifying polymerase activity, amplification buffer; an RNase H activity and the probe and under conditions where the RNA sequences within the probe can form a RNA:DNA heteroduplex with the complimentary DNA sequences in the PCR fragment of the target sequence; and
(e) detecting a real-time increase in the emission of a signal from the label on the probe, wherein the increase in signal indicates the presence of the target DNA in the sample.

31. The method according to claim 30, in which the step (b) further comprises providing a fourth primer which consists of the sequence of the other of the first primer or the second primer, except at least one internal region of 1-30 nucleotides are corresponding ribonucleotides, wherein the third primer has a structure of 5′-{DNA₁-RNA}n-DNA₂-3′, wherein n is an integer of 1-5.

32. The method according to claim 30, in which the step (c) further comprises providing a second probe which has the sequence of the 5′-end region of 5-50 nucleotides of the other of the first primer or the second primer, except at least one internal region of 1-30 nucleotides are corresponding ribonucleotides, said second probe comprising a detectable label.

33. The method according to claim 30, wherein the amplifying polymerase activity is an activity of a thermostable DNA polymer.

34. The method according to claim 30, wherein the RNase H activity is the activity of a thermostable RNase H.

35. A kit for detecting a target sequence in a sample, comprising:
(a) a first primer substantially complementary to a first region of a target sequence;
(b) a second primer substantially complementary to a complementary sequence of a second region of the target sequence;
(c) a third primer of the sequence of one the first primer or the second primer, except at least one internal region of 1-30 nucleotides are corresponding ribonucleotides, wherein the third primer has a structure of 5′-{DNA₁-RNA}n-DNA₂-3′, wherein n is an integer of 1-5;
(d) a first probe which has the sequence of the 5′-end region of 5-50 nucleotides of one of the first primer or the second primer, except at least one internal region of 1-30 nucleotides are corresponding ribonucleotides, said first probe comprising a detectable label;
(e) an amplifying polymerase activity, and
(f) an RNase H activity.

36. The kit according to claim 35, which further comprises a fourth primer which consists of the sequence of the other of the first primer or the second primer, except at least one internal region of 1-30 nucleotides are corresponding ribonucleotides, wherein the third primer has a structure of 5′-{DNA₁-RNA}n-DNA₂-3′, wherein n is an integer of 1-5.

37. The kit according to claim 35, which further comprises a second probe which has the sequence of the 5′-end region 1 of 5-50 nucleotides of the other of the first primer or the second primer, except at least one internal region of 1-30 nucleotides are corresponding ribonucleotides, said second probe comprising a detectable label.

38. The kit according to claim 35, wherein the detectable label on the probe is a fluorescent label.

39. The kit according to claim 35, wherein the amplifying polymerase activity is an activity of a thermostable DNA polymer.
